(19) United States
(12) Patent Application Publication
(10) Pub. No.: US 2005/0064031 A1
(43) Pub. Date: Mar. 24, 2005

(54) Combination contraceptive, kits that contain the latter, and a method that uses the latter

(76) Inventors: Klaus Stockemann, Berlin (DE); Ulrich Koch, Berlin (DE); Kristof Chwalisz, Berlin (DE)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 10/982,799

(22) Filed: Nov. 8, 2004

Related U.S. Application Data
(63) Continuation of application No. 09/392,728, filed on Sep. 9, 1999, now abandoned.
Application No. 09/392,728 is a continuation of application No. 08/698,990, filed on Aug. 16, 1996, now abandoned.

(30) Foreign Application Priority Data
Aug. 17, 1995 (DE) 195 31 936.2

(51) Int. Cl. A61K 31/56; A61K 9/20

(52) U.S. Cl. 424/464; 514/170

(57) ABSTRACT

This invention relates to a multi-phase combination preparation that contains at least 28 daily dosage units: with a first phase of at least 21 initial daily dosage units, containing a competitive progesterone antagonist in a dosage which inhibits ovulation during the first above-named phase; and a second phase of 5 to 28 daily dosage units, in which each dosage unit of this second phase contains a gestagen, as well as a corresponding package (contraceptive kit) that contains this combination preparation and a contraceptive method which uses the combination preparation above.

### BACKGROUND OF THE INVENTION

It is known that competitive progesterone antagonists (antigestagens=AG's), such as, e.g., RU 486 (mifepristones; 11β-[(4-dimethylamino)phenyl]-17β-hydroxy-17α-(1-propinyl)estra-4,9-dien-3-one) are able to inhibit ovulation in various animal species and in women [1) Uilenbroek J. TH. J (1991): Hormone Concentrations and Ovulatory Response in Rats Treated with Antiprogestagens. Journal of Endocrinology, 129, 423-429; 2) Danforth R. et al. (1989): Contraceptive Potential of RU 486 by Ovulation Inhibition: III. Preliminary Observations on Once Weekly Administration, Contraception, 40/2, 195-200; 3) Kekkonen R. et al. (1990): Interference with Ovulation by Sequential Treatment with the Antiprogestin RU 486 and Synthetic Progestin. Fertility and Sterility, 53/4, 747-750; 4) Ledger W. L. et al. (1992): Inhibition of Ovulation by Low Dose Mifepristone (RU 486). Human Reproduction, 7/7, 945-950; 5) Nieman L. K. et al. (1987): The Progesterone Antagonist RU 486: A New Potential New Contraceptive Agent. The New England Journal of Medicine, 316/4, 187-1991].

It is known that the implantation of a fertilized egg can also be prevented by AG's (implantation inhibition); [6) Glassier A. et al. (1992): Mifepristone (RU 486) Compared with High Dose Estrogen and Progesterone for Emergency Postcoital Contraception: New England Journal of Medicine, 8/15; 1041; 7) Puri C. P. et al. (1990): Effects of a Progesterone Antagonist, Ilopristone, On Induction of Menstruation, Inhibition of Nidation, and Termination of Pregnancy in Bonnet Monkeys. Biology of Reproduction, 43, 437-443; 8) Ishwad P. C. et al. (1993): Treatment with a Progesterone Antagonist ZK 98 299 Delays Endometrial Development without Blocking Ovulation in Bonnet Monkeys. Contraception, 48, 57-70; 9) Batista M. C. et al. (1992): Delayed Endometrial Maturation Induced by Daily Administration of the Antiprogestin RU 486: A Potential New Contraceptive Strategy. Am. J. Obstet. Gynecol. 167/1, 60-65].

These findings indicate that antigestagens can be used as contraceptives based on inhibition of ovulation or of implantation.

In addition, for gynecological applications, initial clinical studies have shown that AG's can be used for the treatment of endometriosis and leiomyomata uteri [10) Kettel L. M. et al. (1991): Endocrine Responses to Long-Term Administration of the Antiprogesterone RU 486 in Patients with Pelvic Endometriosis. Fertility and Sterility, 56/3, 402-407; 11) Kettel L. M. et al. (1993): Long-Term, Low-Dose RU 486 in the Treatment of Endometriosis. Meeting of the Society of Gynecological Investigation 1993, Abstract S-136; 12) Murphy A. A. et al. (1993): Regression of Uterine Leiomyomata in Response to the Antiprogestin RU 486, J. Clin. Endocrinol. Metab., 76/2, 513-517].

The findings of these studies indicate that in the case of chronic treatment with AG's over the entire menstrual cycle, but also in the case of treatment during specific cycle phases with AG's, the elimination of the progesterone action during the luteal phase of the cycle can lead to displacement or lengthening of the cycle with cessation of menstruation (amenorrhea) or weakened menstruation [8), 9), 10)].

Both the endometrium and the hypothalamic-hypophyseal-ovarian axis are suitable as target organs for a contraceptive preparation with estrogens, gestagens, or their antagonists.

Estrogens alone can inhibit the increase of gonadotropin that is necessary for ovulation with a negative feedback mechanism. In the normal cycle, the increase in estrogen during the follicle phase and shortly before ovulation modulates the increase in gonadotropin that is necessary for ovulation. Estrogens at high dosage can also interfere with the nidation of the fertilized egg (implantation).

The role of progesterone in ovulation is still not understood exactly.

Continuous administration of gestagens results in inhibition of the secretion of gonadotropin. In the normal cycle, progesterone seems to play an important role in ovulation. Recent studies have shown that the slight rise in progesterone shortly before ovulation seems to be of decisive importance.

Administering a competitive progesterone antagonist (e.g., RU 486) at this time blocks ovulation. Simultaneous administration of progesterone again cancels this effect [19) Batista M. C. et al., 1992: Evidence for a Critical Role of Progesterone in the Regulation of the Midcycle Gonadotropin Surge and Ovulation. Journal of Clinical Endocrinology and Metabolism 74: 565-570]. In addition to these effects, it is known that progesterone is involved in the enzymatic dissolution of the follicle wall and thus plays a role for ovulation on the level of the ovary.

In addition, progesterone is of decisive importance in the cycle of the woman for secretory conversion of the endometrium and for preparing for the implantation of the fertilized egg.

Based on this knowledge, various contraceptives are conceivable. In conventional oral contraceptives, which consist, in most cases, of a combination of an estrogen and gestagen, the two components together result in inhibition of ovulation.

As an alternative to this, there are contraceptives that contain only one progesterone component (progestagen only pill ═POP); the latter are mainly used when side effects occur with conventional contraceptives (estrogen and gestagen) or in women who are at elevated risk of thrombosis for which the estrogen component is believed to be responsible [20) Gerstman et al., 1991, Oral Contraceptive Estrogen Dose and the Risk of Deep Venous Thromboembolic Disease. Am. J. Epidemiol. 133: 32-37]. These POP's also have drawbacks, however. Thus, with POP's, a considerable increase in intracyclic menstrual bleeding occurs [21) Broome M., Fortherby K. (1990); Clinical Experience with the Progesterone-Only Pill. Contraception 42: 489-494]. Also, the principle of action is different than that of conventional OC's; ovulation inhibition does not occur in all women. In addition to a possible role of estrogens, progesterone is now also considered to play an important role in the pathogenesis of breast cancer [22) King R. J. B. (1990); A Discussion on the Roles of Estrogen and Progestin in Human Mammary Carcinogenesis. J. Ster. Biochem. Molec. Biol. 39: 8111-81; 23) Paul C., Skeeg D. C. G., Spears G. F. S. (1989): Depot Medroxyprogesterone (Depo-provera) and Risk of Breast Cancer. Br. Med. J. 299: 759-762]. Although this is not yet definitively proven, a few studies with gestagen preparations so indicate.

For the above-mentioned reasons, it is therefore desirable to reduce the dosages of estrogen and gestagen in contraceptives.

For the use of a competitive progesterone antagonist in contraception, three possibilities are considered, namely its use as an ovulation inhibitor, as a postcoital pill, or as a luteal contraceptive.

Preparations for contraception with antigestagens for the purpose of inhibiting ovulation with subsequent gestagen administration in the treatment cycle have been described [Kekkonen et al., (1990) Fertility and Sterility, 53(4): 747-750 Interference with Ovulation by Sequential Treatment with the Antiprogesterone RU 486 and Synthetic Progestin, Bergink WO 94/04156]. Kekkonen et al. describes a discontinuous regimen in which 25 mg of mifepristone is administered daily from day 1-14 of the menstrual cycle, followed by 3 mg of the gestagen norethisterone on days 15-24, followed by a 5-day placebo administration. This regimen was used over 3 cycles. With the regimen described there, it was not possible to suppress the serum concentrations for FSH and LH. In addition, when gestagen was administered in the first two cycles, there was evidence that ovulation and maturation of follicles had occurred. These results indicate that the regimen that is described by Kekkonen et al. does not provide for sufficient contraceptive safety and thus can result in unintentional pregnancy.

The regimen that is disclosed in the patent application by Bergink comprises uninterrupted application for first at least 5 days and at most 20 days of an antigestagen and then for at least 10 days, but at most 25 days, of a gestagen. The preferred embodiment comprises the sequential administration for 14 days of an antigestagen and then the administration of a gestagen for 14 days. Also, with such a regimen, ovulation is not inhibited with the desired reliability since the antigestagen phase is not long enough.

### SUMMARY OF THE INVENTION

This invention relates to a multi-phase combination preparation that contains at least 28 daily dosage units:
with a first phase of at least 21 initial daily dosage units, containing a competitive progesterone antagonist in one dosage, which inhibits ovulation during the first above-named phase; and a second phase of 5 to 28 daily dosage units, in which each dosage unit of this second phase contains a gestagen, as well as a corresponding package (contraceptive kit) that contains this combination preparation, and a contraceptive method that uses the combination preparation above.

The object of this invention was to provide a product for contraception that contains an antigestagen for ovulation inhibition and induction of amenorrhea, with sequential administration of a gestagen in order to prepare for bleeding, which offers the necessary contraceptive safety by reliably inhibiting ovulation of the first cycle and which in addition makes it possible to select the cycle length in a variable manner.

Upon further study of the specification and appended claims, further objects and advantages of this invention will become apparent to those skilled in the art.

This object is achieved by providing the multi-phase combination preparation that is mentioned in the introduction.

In preferred embodiments of the multi-phase combination preparation that is described in the introduction, the first phase comprises:
21 to 28,
at most 77,
28
28 plus 7 or 28 plus a multiple of 7 initial daily dosage units, and the second phase:
7 to 14 daily dosage units.

In an especially preferred embodiment of the multi-phase combination preparation, the first phase comprises 21 initial daily dosage units, and the second phase comprises 7 daily dosage units.

In another preferred embodiment of the multi-phase combination preparation, the first phase comprises 23 or 24 initial daily dosage units, and the second phase comprises 8, 7 or 6 daily dosage units, to ensure that the total number of daily dosage units of the first and the second phases adds up to 30 or 31 days (so-called monthly packaging).

In another especially preferred embodiment of the multi-phase combination preparation, the first phase comprises 70 initial daily dosage units, and the second phase comprises 14 daily dosage units.

In another especially preferred embodiment of the multi-phase combination preparation, the first phase comprises 63 initial daily dosage units, and the second phase comprises 7 daily dosage units.

This invention further comprises a contraceptive kit that contains at least 28 daily dosage units with:
a first phase of at least 21 initial daily dosage units that contain in every individual dosage unit a competitive progesterone antagonist in a dosage that inhibits ovulation during the first above-named phase; and a second phase of 5 to 28 separate daily dosage units, in which each dosage unit of the second phase contains a gestagen.

Preferred embodiments of the contraceptive kit are distinguished in that the first phase:
21 to 28,
at most 77,
28,
28 plus 7 or 28 plus a multiple of 7 of initial daily dosage units, and the second phase;
7 to 14 daily dosage units.

In an especially preferred embodiment of the contraceptive kit, the first phase comprises 21 initial daily dosage units, and the second phase comprises 7 daily dosage units.

In another especially preferred embodiment of the contraceptive kit, the first phase comprises 23 or 24 initial daily dosage units, and the second phase comprises 7 or. 6 daily dosage units, to ensure that the total number of the daily dosage units of the first and second phase adds up to 30 or 31 days (so-called monthly packaging).

In another especially preferred embodiment of the contraceptive kit, the first phase comprises 70 initial daily dosage units, and the second phase comprises 14 daily dosage units.

In another especially preferred embodiment of the contraceptive kit, the first phase comprises 63 initial daily dosage units, and the second phase comprises 7 daily dosage units.

In another embodiment of the contraceptive kit according to the invention, dosage units of the first and/or second phase come to some extent in periodically repeating subunits that are separated from one another physically and/or by other markings. This makes it possible to handle the initiation of withdrawal bleeding in a variable manner.

The daily dosage units preferably come in subunits that can be separated from one another by perforations or other devices that are suitable for separation.

They are contained in an especially preferred embodiment of the invention in separate subunits in 7 dosage units apiece.

### BRIEF DESCRIPTION OF THE DRAWINGS

Various other objects, features and attendant advantages of the present invention will be more fully appreciated as the same becomes better understood when considered in conjunction with the accompanying drawings, in which like reference characters designate the same or similar parts throughout the several views, and wherein:

FIG. 1 shows three 21-day blisters containing dosage units of an antigestagen;

FIG. 2 shows a blister containing dosage units of a gestagen, separated by perforations into subunits;

FIG. 3 shows a contraceptive kit containing a 21 day antigestagen blister and a 21 day gestagen blister, wherein the latter are separated by perforations into subunits; and

FIG. 4 shows a contraceptive kit containing a different configuration of blisters of antigestagen and gestagen, wherein both phases are separated by perforations into subunits.

Subunits are defined as any means of dividing up or subdividing, such as, for example, a blister, in several blisters, or else also subdivision within a blister; all possible means of dividing up or subdividing in between are conceivable.

The dividing up of the contraceptive kit facilitates the variable handling of the initiation of withdrawal bleeding, already described previously; the separated subunits can be separated from one another by the perforations, and the kit can thus be adapted to the individual regimen.

The invention further relates to a method for contraception in the case of female mammals that consists of at least 28 days of sequential administration:
of at least 21 initial daily dosage units during a first phase, containing a competitive progesterone antagonist in a dosage that inhibits ovulation during the first above-named phase, and a second phase of 5 to 28 daily dosage units in which each dosage unit of this second phase contains a gestagen.

As competitive progesterone antagonists according to this invention, all compounds are considered that themselves or their metabolic products block the action of the progesterone on its receptor. As examples of typical competitive progesterone antagonists, the following can be mentioned here:
17α-ethinyl-17β-hydroxy-11β-(4-methoxyphenyl)estra-4,9-dien-3-one (Steroids 37 (1981), 361-382),
11β-(4-acetylphenyl)-17β-hydroxy-17α-(1-propinyl)estra-4,9-dien-3-one (EP-A 0 190 759),
(Z)-11β-(4-acetylphenyl)-17 μl-hydroxy-17α-(3-hydroxy-1-propenyl)estra-4,9-dien-3-one,
11β-(4-dimethylaminophenyl)-17β-hydroxy-17α-(1-propinyl)estra-4,9-dien-3-one (RU 486),
(Z)-9,11α-dihydro-17β-hydroxy-17α-(3-hydroxy-1-propenyl)-6′-(3-pyridinyl)-4′H-naphth[3′,2′,1′:10,9,11]estra-4,9(11)-dien-3-one,
(Z)-11β-[4-(dimethylamino)phenyl]-17β-hydroxy-17α-(3-hydroxy-1-propenyl)-estr-4-en-3-one,
4′,5′-dihydro-11β-[4-(dimethylamino)phenyl]-6β-methylspiro[estra-4,9-dien-17β,2′(3′H)-furan]-3-one,
4′,5′-dihydro-11β-[4-(dimethylamino)phenyl]-7β-methylspiro[estra-4,9-dien-17β,2′(3′H)-furan]-3-one, 11β-(4-acetylphenyl)-19,24-dinor-17,23-epoxy-17α-chola-4,9,20-trien-3-one, as well as
19,11β-bridged steroids from EP-A-0 283 428 and
10β-H steroids from EP-A-0 404 283.

In an especially preferred embodiment of the invention, the competitive progesterone antagonist is the compound (Z)-11β-[4-(dimethylamino)phenyl]-17β-hydroxy-17α-(3-hydroxy-1-propenyl)estr4-en-3-one or a compound that is comparable in its profile.

This list is not exhaustive; other competitive progesterone antagonists described in the above-mentioned publications as well as those from publications that are not mentioned here are also suitable.

The competitive progesterone antagonists are tested on female cynomolgus monkeys for their ability to inhibit ovulation. For this purpose, the animals received the respective test substance daily from day 2 to day 22 of the menstrual cycle administered, e.g., orally. By determining the serum progesterone value especially on day 20 or 22 of the menstrual cycle or by ultrasound studies, the ovulation-inhibiting action of the respective test substance is determined.

In the contraceptively active combination preparation according to the invention, the competitive progesterone antagonist is to be administered in a dose which inhibits ovulation and induces amenorrhea. For, e.g., RU 468 this is 2 mg/woman per day, while for the other competitive progesterone antagonists the amounts are in the range of 0.01-30 mg/woman per day.

The threshold dose of an ovulation-inhibiting competitive progesterone antagonist is determined in women in clinical trials by determining the serum progesterone values or by ultrasound studies.

After the endometrium phase which is receptive in the normal cycle and is inhibited by competitive progesterone antagonists, the endometrium is prepared by gestagen—corresponding to the normal cycle in the luteal phase—for bleeding that is induced by the progesterone antagonist that is again administered after gestagen is given.

As gestagens, according to this invention, all compounds are suitable that are suitable for use in oral contraceptives because of their gestagenic activity. A list of such compounds is found in B. Runnebaum et al., “Female Contraception: Update and Trends,” Springer-Verlag, Berlin, 1988, pages 64-90, 109-121, 122-128 and 129-140.

In all embodiments of the invention, the gestagen is preferably selected from the following group of compounds:
gestodene,
progesterone,
levonorgestrel,
cyproterone acetate,
chlormadinone acetate,
drospirenone (dihydrospirorenone),
norethisterone,
norethisterone acetate,
norgestimate,
desogestrel,
3-ketodesogestrel,
dienogest
   or a mixture of the above.

In an especially preferred embodiment, the gestagen is contained in a daily dosage of:
0.02-0.6 mg of levonorgestrel,
0.02-2.0 mg of cyproterone acetate,
0.01-0.3 mg of gestodene,
0.02-0.3 mg of desogestrel
   or in a bioequivalent dosage of another gestagen.

In an especially preferred embodiment, the gestagen gestodene is contained in a dosage of 0.02 to 0.075 mg.

In cases where the antigestagen is administered in an ovulation-inhibiting dose and in a dose that induces amenorrhea, endogenic progesterone is avoided; likewise the endogenic estrogen is reduced, but not completely suppressed [10) Kettel L. M. et al.]. Ovulation is inhibited, and the endometrium is not converted to an endometrium that can bleed, which leads to amenorrhea.

The blocking of progesterone can lead to a breast carcinoma-protective effect. The fact that antigestagens inhibit the proliferation of normal mammary gland tissue and can inhibit the growth of breast tumors that are induced experimentally in animals has been described [Spitzer et al., (1995): Antiprogestin Inhibit Growth and Stimulate Differentiation in the Normal Mammary Gland. Journal of Cellular Physiology 164: 1-8; Schneider et al. (1989). Antitumor Activity of the Progesterone Antagonists ZK 98 299 and RU 38.486 in the Hormone-dependent MXT Mammary Tumor Model of the Mouse and the DMBA- and NMU-Induced Mammary Tumor Model of the Rat. Eur. J. Cancer Clin. Oncol. 25, 691-701]. In addition, it has been shown that if rats are treated with progesterone antibodies or with onapristone before the administration of a carcinogen, the development of breast tumors can be inhibited.

After the respective administration of the antigestagen, the way is paved for bleeding with the aid of a gestagen. Since complete suppression of estrogens does not occur under antigestagen treatment (the levels are comparable to those of the middle follicle phase [10) Kettel L. M. et al.]), after ovulation inhibition a gestagen can transform the endometrium in the receptive phase (only in this phase could an implantation occur—if it is not inhibited) by the antigestagen and convert it into a secretorily active endometrium and prepare it for bleeding that corresponds to natural menstruation, which after gestagen administration is induced by continued antigestagen treatment.

The subsequent continuing treatment with a competitive progesterone antagonist (in ovulation-inhibiting dose) simulates the natural drop in progesterone (progesterone blocking) and triggers menstruation, in which portions of the endometrium are shed. That this is possible after antigestagen treatment—also in the presence of progesterone—was shown [Nieman L. K. et al. (1987); The Progesterone Antagonist RU 486; A Potential New Contraceptive Agent. The New England Journal of Medicine, 316/4, 187-191; Craxatto H. B. et al. (1985). The Demonstration of Antiprogestin Effects of RU 486 When Administered to the Human During hCG-Induced Pseudopregnancy. In Baulieu E. E. and Segal S. J. (eds.). The Antiprogestin Steroid RU 486 and Human Fertility Control. Plenum Press, New York, pp. 263-269; Kekkonen R. et al. (1993): Sequential Regimen of the Antiprogesterone RU 486 and Synthetic Progestin for Contraception. Fertility and Sterility 60: 610-615].

In the normal cycle, two phases are distinguished: the proliferation phase (follicle phase) and the secretion phase (luteal phase). In the follicle phase, estrogen-induced development of the secretory glands in the endometrium occurs in the normal menstrual cycle, while in the luteal phase, the gestagen (progesterone) induces the secretory activity of the glands.

According to this invention, the progesterone antagonist serves to inhibit the ovulation; but it also results in an inhibition of both the development of endometrial glands as early as during the follicle phase and the secretory conversion of glands in the luteal phase, which is essential for successful implantation of the fertilized egg.

Sequential treatment with gestagen ensures suitable cycle control.

The undesirable effects of possible monotherapy with a competitive progesterone antagonist (chronic amenorrhea and stimulation of the endometrium) can be prevented with the proposed composition.

In diagrams 1, 2, 3 and 4, by way of example, different possible configurations of the composition are depicted.

In the product according to the invention, the gestagen that is sequentially administered to the competitive progesterone antagonist is provided for administration at the earliest starting on the 21st day after the first administration of the competitive progesterone antagonist.

The number of dosage units of the competitive progesterone antagonist to be administered as well as the number of gestagen-containing dosage units that are subsequently to be administered daily can be selected in such a way that the menstruation that is triggered by the administration of gestagen corresponds in time to menstruation in an untreated cycle (diagram 1).

The composition according to the invention can also contain the dosage units of gestagen that are to be administered sequentially to the competitive progesterone antagonist, arranged in such a way that they are provided at the latest after 2×28-day plus 14-day administration of the progesterone antagonist, whereby the administration of gestagen is 14 days (corresponding to the period of a total of three months) (diagram 5).

Between these two limits (competitive progesterone antagonist over a period of 21 or 2×28 days plus 14 days), all conceivable cases are possible, thus, e.g., 3×21 days-administration of the competitive progesterone antagonist, then 7 days of gestagen administration (diagram 2) or else also the administration of 23 or 24 days of the competitive progesterone antagonist, then 7 or 6 days of gestagen administration (diagram 3).

Another possible variant calls for the administration of 2×28 days plus 21 days of the competitive progesterone antagonist, with subsequent 7-day gestagen administration (diagram 4).

Without further elaboration, it is believed that one skilled in the art can, using the preceding description, utilize the present invention to its fullest extent. The following preferred specific embodiments are, therefore, to be construed as merely illustrative and not limitative of the remainder of the disclosure in any way whatsoever.

In the foregoing and in the following examples, all temperatures are set forth uncorrected in degrees Celsius; and, unless otherwise indicated, all parts and percentages are by weight.

The entire disclosures of all applications, patents and publications, cited above and below, including DE 195 31 936.2, are hereby incorporated by reference.

### EXAMPLES

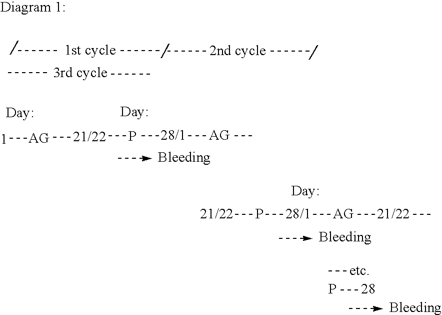

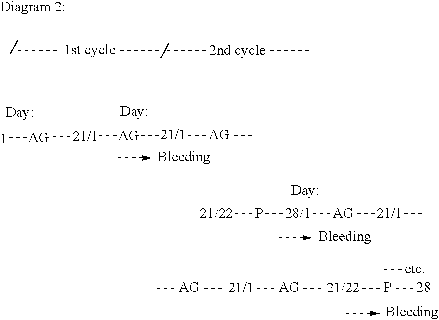

AG stands for “competitive progesterone antagonist” and P stands for “gestagen”

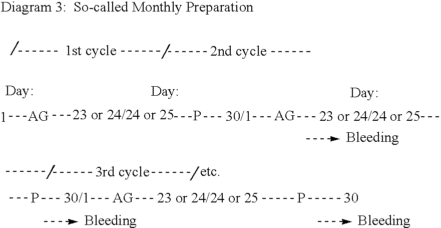

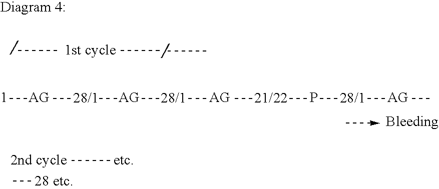

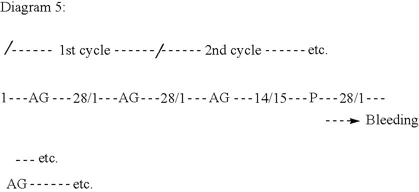

Two studies in the Netherlands and Germany, France and in the United Kingdom have revealed that many women would prefer to have their menstruation less often. Hygiene problems, monthly pains, and emotional instability were mentioned as the main reasons. The existing invention makes it possible for women to determine the length of their cycles themselves. According to this invention, it is possible to extend withdrawal bleeding to any day beyond day 84. Since women have different preferences, their needs can be met with this invention using, e.g., a 2-month or else even a ¼-year cycle, and thus the side effects that accompany menstruation are reduced accordingly.

The combination preparations according to the invention or kits that contain the latter are especially suitable for an extended menstrual cycle, since alteration or extension of the cycle with cessation of the monthly bleeding (amenorrhea) is induced by the chronic administration of antigestagens over the entire cycle, owing to the elimination of the progesterone action during the luteal phase.

The special division of the contraceptive kit into subunits makes it possible to handle the initiation of withdrawal bleeding in variable ways. For example, perforations make it easy to separate the individual subunits from one another and thus to adapt them optimally to the individual regimen.

With the aid of the contraceptive kit, women can determine the length of their menstrual cycles themselves.

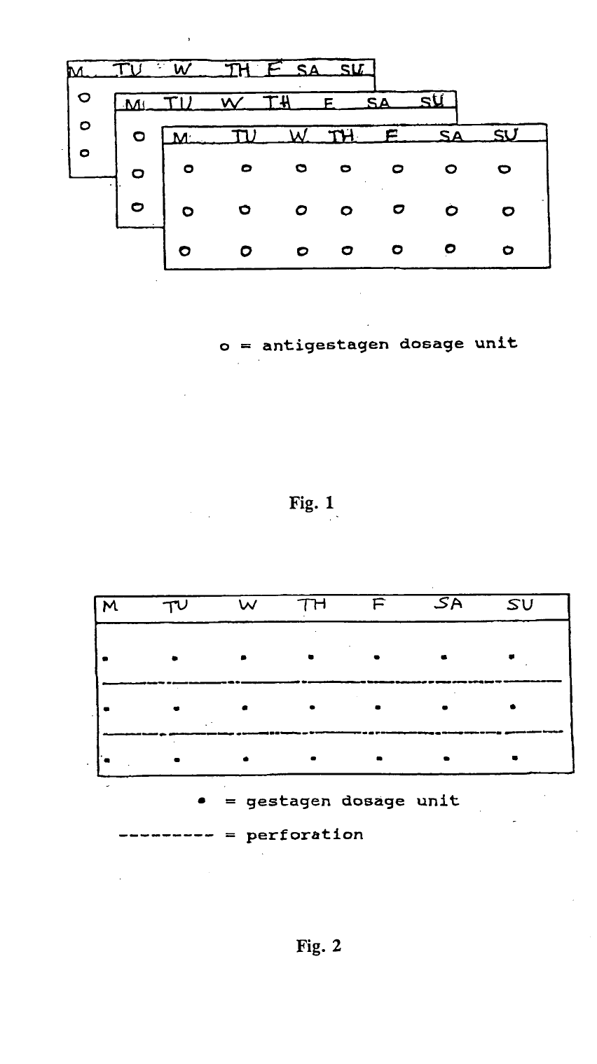

The woman would take, for example, the contents of three 21-day blisters that contain only dosage units of an antigestagen (FIG. 1) and the contents of a blister which contains only dosage units of a gestagen.

The number of gestagen-containing dosage units that are required for the transformation of the endometrium can be separated as subunits (FIG. 2).

If the woman decides to follow, for example, a 4-week regimen, then she first takes the antigestagen blister with, e.g., 21 coated tablets and then takes, e.g., 7 days of gestagen dosage units.

If she should opt for a longer cycle with less bleeding, then she can consume in succession two or three antigestagen blisters or subunits with 21 coated tablets that contain a competitive progesterone antagonist, and then the number of gestagen-containing dosage units that is required for the transformation of the endometrium (e.g., 7 days) is detached as a subunit and taken.

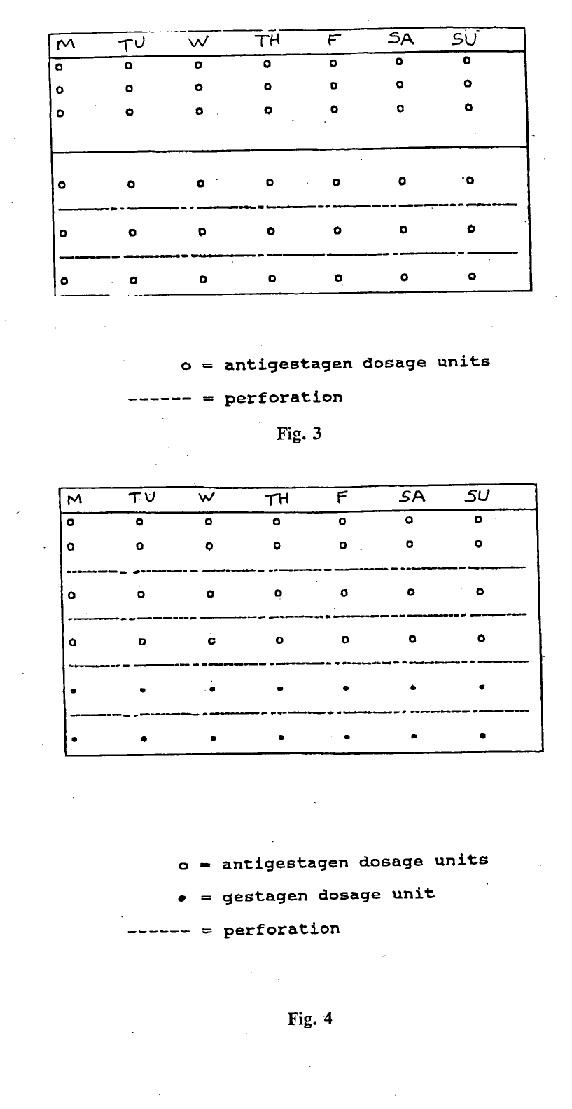

Another configuration of the contraceptive kit, by way of example, could contain an antigestagen blister, as depicted in FIG. 3, and a gestagen blister, as shown in FIG. 2. Here there are 42 dosage units of an antigestagen in a blister; here the first 21 antigestagen-containing dosage units are not separated from one another by perforations, but the subsequent 3×7 antigestagen-containing dosage units can be separated from one another by perforations (FIG. 3).

If the woman opts for, e.g., a 5-week cycle, she detaches 21+7 antigestagen-containing dosage units from the blister and takes them in succession. If the woman should opt for, for example, a 6-week cycle, however, then she detaches 21+7+7 antigestagen-containing dosage units from the blister and takes them in succession. Then she detaches as a subunit the number of gestagen-containing dosage units (e.g., 7 days) that is required in each case for the transformation of the endometrium from a gestagen blister as shown in FIG. 2 and takes these. In another embodiment of the contraceptive kit, as depicted in FIG. 4, the antigestagen dosage units and the gestagen dosage units are in a blister.

The administration of the competitive progesterone antagonists according to this invention can be done locally, topically, enterally, transdermally, or parenterally.

For the preferred oral administration, especially tablets, coated tablets, capsules, pills, suspensions, or solutions are suitable, which can be produced in the usual way with the additives and vehicles that are commonly used in galenicals.

The formulation of the competitive progesterone antagonist is done analogously to what is known, for, e.g., RU 486.

For local or topical application, for example, vaginal suppositories, vaginal gels, implants, vaginal rings, or transdermal systems such as skin patches are suitable.

A dosage unit contains about 2 mg of 11β-[4-(dimethylamino)phenyl]-17β-hydroxy-17α-(1-propinyl)estra4,9-dien-3-one (RU 486) or a biologically equivalent amount of another competitive progesterone antagonist.

If the administration of the competitive progesterone antagonist to be used according to the invention is carried out by an implant, a vaginal ring or a transdermal system, these administration systems must be designed in such a way that a dose with action equivalent to the daily oral dose for the respective form of administration of the competitive progesterone antagonist is released by them daily (comparable serum concentrations).

The dose of a competitive progesterone antagonist that is to be administered according to the invention lies in an ovulation-inhibiting as well as non-abortion-inducing dose range of the progesterone antagonist in question and is sufficient to trigger amenorrhea.

One-time administration is also to mean that when using an administration system that continuously releases competitive progesterone antagonists, 0.01-30 mg each per day is released.

The product according to the invention can also be designed such that the administration of the individual dosage units of the competitive progesterone antagonist can be done every 4 to every 10 days, specifically beginning on any day before the ovulation time during the first administration cycle. As a result, contraceptive reliability is already ensured in the first administration cycle (Bygdeman).

The time intervals between the administrations of individual dosage units are preferably to be constant in this case.

In another embodiment of the product according to the invention, the administration of the respective dosage units of the competitive progesterone antagonist is done in such a way that the latter are administered once per week respectively on the same day of the week, for example, on a Monday (“Monday pill”).

The dose of the competitive progesterone antagonist in the case of administration at 4- to 7-day intervals must be selected in such a way that, expressed in terms of a daily dosage, the respective dosage contains 4× to 7× active ingredient, so that an amount of active ingredient that corresponds to the daily dosage is released per day.

High intake reliability is ensured by the weekly administration cycle that falls on the same day every week.

The product according to the invention can also provide for the administration of the individual dosage units of the competitive progesterone antagonist daily, every second or every third day; and the amount of active ingredient per dosage must then be increased accordingly.

In the product according to this invention, the gestagen is present in a dosage form that is suitable for oral administration, namely as a tablet, coated tablet, capsule or pill.

In this case, the formulation of the gestagen is done in a way analogous to preparing gestagens for hormonal contraception with use of the adjuvants that are commonly used for this purpose.

Determining equivalent-action dose amounts of various gestagens is done according to known methods; further details are found in, for example, the two articles “Probleme der Dosisfindung: Sexualhormone [Problems of Dose-Finding: Sex Hormones]”; F. Neumann et al. in “Arzneimittelforschung [Drug Research]” 27, 2a, 296-318 (1977) as well as “Aktuelle Entwicklungen in der hormonalen Kontrazeption [Current Developments in Hormonal Contraception]”; H. Kuhl in “Gynakologe [Gynecologists]” 25: 231-240 (1992).

The preceding examples can be repeated with similar success by substituting the generically or specifically described reactants and/or operating conditions of this invention for those used in the preceding examples.

From the foregoing description, one skilled in the art can easily ascertain the essential characteristics of this invention, and without departing from the spirit and scope thereof, can make various changes and modifications of the invention to adapt it to various usages and conditions.

1. A multi-phase combination preparation comprising at least 28 daily dosage units, comprising
a first phase of at least 21 initial daily dosage units, each comprising a competitive progesterone antagonist in an amount effective to inhibit ovulation during the first phase; and
a second phase of 5 to 28 daily dosage units, each dosage unit of this second phase comprising a gestagen.

2. A multi-phase combination preparation of claim 1, wherein the first phase comprises 21 to 27 initial daily dosage units.

3. A multi-phase combination preparation of claim 1, wherein the first phase comprises at least 28 and at most 77 initial daily dosage units.

4. A multi-phase combination preparation of claim 1, wherein the first phase comprises 28 initial daily dosage units.

5. A multi-phase combination preparation of claim 1, wherebin the second phase comprises 7 to 14 daily dosage units.

6. A multi-phase combination preparation of claim 1, wherein the first phase comprises 21 initial daily dosage units and the second phase comprises 7 daily dosage units.

7. A multi-phase combination preparation of claim 1, wherein the first phase comprises 23 or 24 initial daily dosage units and the second phase comprises 8, 7 or 6 daily dosage units, whereby the total number of daily dosage units of the first and the second phases is 30 or 31.

8. A multi-phase combination preparation of claim 1, wherein the first phase comprises 70 initial daily dosage units and the second phase comprises 14 daily dosage units.

9. A multi-phase combination preparation of claim 1, wherein the first phase comprises 63 initial daily dosage units and the second phase comprises 7 daily dosage units.

10. A multi-phase combination preparation of claim 1, wherein the competitive progesterone antagonist is selected from:
17α-ethinyl-17β-hydroxy-11β-(4-methoxyphenyl)estra-4,9-dien-3-one,
11β-(4-acetylphenyl)-17β-hydroxy-17α-(1-propinyl)estra-4,9-dien-3-one,
(Z)-11β-(4-acetylphenyl)-17β-hydroxy-17α-(3-hydroxy-1-propenyl)estra4,9-dien-3-one,
11β-(4-dimethylaminophenyl)-17β-hydroxy-17α-(1-propinyl)estra-4,9-dien-3-one,
(Z)-9,11α-dihydro-17β-hydroxy-17α-(3-hydroxy-1-propenyl)-6′-(3-pyridinyl)-4′H-naphth[3′,2′,1′:10,9,11]estra-4,9(11)-dien-3-one,
(Z)-11β-[4-(dimethylamino)phenyl]-17β-hydroxy-17α-(3-hydroxy-1-propenyl)-estr-4-en-3-one,
4′,5′-dihydro-11β-[4-(dimethylamino)phenyl]-6β-methylspiro[estra-4,9-dien-17β,2′(3′H)-furan]-3-one,
4′,5′-dihydro-11β-[4-(dimethylamino)phenyl]-7β-methylspiro[estra-4,9-dien-17β,2′(3′H)-furan]-3-one, or
11β-(4-acetylphenyl)-19,24-dinor-17,23-epoxy-17α-chola-4,9,20-trien-3-one, or a mixture thereof.

11. A multi-phase combination preparation of claim 1, wherein the gestagen is selected from:
gestodene,
progesterone,
levonorgestrel,
cyproterone acetate,
chlormadinone acetate,
drospirenone (dihydrospirorenone),
norethisterone,
norethisterone acetate,
norgestimate,
desogestrel,
3-ketodesogestrel, or
dienogest,
or a mixture thereof.

12. A multi-phase combination preparation of claim 1, wherein the daily dosage of the gestagen is:
0.02-0.6 mg of levonorgestrel,
0.02-2.0 mg of cyproterone acetate,
0.01-0.3 mg of gestodene, or
0.02-0.3 mg of desogestrel,
or a bioequivalent dosage of another gestagen.

13. A multi-phase combination preparation of claim 1, wherein the gestagen gestodene is in a dosage of 0.02 to 0.075 mg.

14. A multi-phase combination preparation of claim 1, wherein the competitive progesterone antagonist(Z)-11β-[4-(dimethylamino)phenyl]-17βB-hydroxy-17α-(3-hydroxy-1-propenyl)estr4-en-3-one is in a dosage of 0.01-5 mg.

15. A contraceptive kit comprising at least 28 daily dosage units, comprising
a first phase of at least 21 initial daily dosage units, each individual dosage unit comprising a competitive progesterone antagonist in an effective amount to inhibit ovulation during the first above-named phase; and
a second phase of 5 to 28 separate daily dosage units, wherein each dosage unit of the second phase comprises a gestagen.

16. A contraceptive kit of claim 15, wherein the first phase comprises 21 to 27 initial daily dosage units.

17. A contraceptive kit of claim 15, wherein the first phase comprises at least 28 and at most 77 initial daily dosage units.

18. A contraceptive kit of claim 15, wherein the first phase comprises 28 initial daily dosage units.

19. A contraceptive kit of claim 15, wherein the second phase comprises 10 to 25 daily dosage units.

20. A contraceptive kit of claim 15, wherein the first phase comprises 21 initial daily dosage units and the second phase comprises 7 daily dosage units.

21. A contraceptive kit of claim 15, wherein the first phase comprises 23 or 24 initial daily dosage units and the second phase comprises 8, 7 or 6 daily dosage units, whereby the total number of daily dosage units of the first and the second phases is 30 or 31.

22. A contraceptive kit of claim 15, wherein the first phase comprises 70 initial daily dosage units and the second phase comprises 14 daily dosage units.

23. A contraceptive kit of claim 15, wherein the first phase comprises 63 initial daily dosage units and the second phase comprises 7 daily dosage units.

24. A contraceptive kit of claim 15, wherein the competitive progesterone antagonist is selected from:
17α-ethinyl-17β-hydroxy-11β-(4-methoxyphenyl)estra-4,9-dien-3-one,
11β-(4-acetylphenyl)-17β-hydroxy-17α-(1-propinyl)estra-4,9-dien-3-one,
(Z)-11β-(4-acetylphenyl)-17β-hydroxy-17α-(3-hydroxy-1-propenyl)estra-4,9-dien-3-one,
11β-(4-dimethylaminophenyl)-17β-hydroxy-17α-(1-propinyl)estra-4,9-dien-3-one,
(Z)-9,11α-dihydro-17β-hydroxy-17α-(3-hydroxy-1-propenyl)-6′-(3-pyridinyl)-4′H-naphth[3′,2′,1′:10,9,11]estra-4,9(11)-dien-3-one,
(Z)-11β-[4(dimethylamino)phenyl]-17α-hydroxy-17α-(3-hydroxy-1-propenyl)-estr-4-en-3-one,
4′,5′-dihydro-1 1β-[4-(dimethylamino)phenyl)-6β-methylspiro[estra-4,9-dien-17β,2′(3′H)-furan]-3-one,
4′,5′-dihydro-11β-[4-(dimethylamino)phenyl]-7β-methylspiro[estra-4,9-dien-17β,2′(3′H)-furan]-3-one, or
11β-(4-acetylphenyl)-19,24-dinor-17,23-epoxy-17α-chola-4,9,20-trien-3-one,
or a mixture thereof.

25. A contraceptive kit of claim 15, wherein the gestagen is selected from:
gestodene,
progesterone,
levonorgestrel,
cyproterone acetate,
chlormadinone acetate,
drospirenone (dihydrospirorenone),
norethisterone,
norethisterone acetate,
norgestimate,
desogestrel,
3-ketodesogestrel, or
dienogest,
or a mixture thereof.

26. A contraceptive kit of claim 15, wherein the daily dosage of gestagen is:
0.02-0.6 mg of levonorgestrel,
0.02-2.0 mg of cyproterone acetate,
0.01-0.3 mg of gestodene, or
0.02-0.3 mg of desogestrel,
or a bioequivalent dosage of another gestagen.

27. A contraceptive kit of claim 15, wherein the gestagen gestodene is in a dosage of 0.02 to 0.075 mg.

28. A contraceptive kit of claim 15, wherein the competitive progesterone antagonist (Z)-11β-[4-(dimethylamino)phenyl]-17β-hydroxy-17α-(3-hydroxy-1-propenyl)estr4-en-3-one is in a dosage of 0.01-5 mg.

29. A contraceptive kit comprising at least 28 daily dosage units, comprising:
a first phase of at least 21 separate initial daily dosage units, comprising a competitive progesterone antagonist in an effective amount to inhibit ovulation during the first above-named phase; and
a second phase of 5 to 28 separate daily dosage units, wherein each dosage unit of the second phase comprises a gestagen,
wherein
the respective dosage units are in periodically repeating subunits separated from one another spatially and/or by other markings,
whereby
the dosage units that are present in the first phase comprise at least 21 daily dosage units in a kit, and
the daily dosage units that are present in the second phase comprise at least 7 daily dosage units.

30. A contraceptive kit of claim 29, wherein the individual subunits can be separated from one another by perforations or other devices suitable for separation.

31. A contraceptive kit of claim 29, wherein the separate subunits each contain 7 dosage units.

32. A contraceptive kit of claim 29, wherein the separate subunits of the first phase each contain 7 dosage units.

33. A method for contraception in a female mammal, comprising administering, in an at least 28-day sequential administration regimen:
a first phase of at least 21 initial daily dosage units comprising a competitive progesterone antagonist in an amount effective to inhibit ovulation during the first above-named phase, and
a second phase of 7 to 28 daily dosage units, wherein each dosage unit comprises a gestagen.

34. A method for contraception of claim 33, wherein during the first phase, 21 to 27 initial daily dosage units are administered.

35. A method for contraception of claim 33, wherein during the first phase, at least 28 and at most 77 initial daily dosage units are administered.

36. A method for contraception of claim 33, wherein during the first phase, 28 initial daily dosage units are administered.

37. A method for contraception of claim 33, wherein during the second phase, 7 to 14 daily dosage units are administered.

38. A method for contraception of claim 33, wherein the first phase comprises 21 initial daily dosage units and the second phase comprises 7 daily dosage units.

39. A method for contraception of claim 33, wherein the first phase comprises 23 or 24 initial daily dosage units and the second phase comprises 8, 7 or 6 daily dosage units, whereby the total number of daily dosage units of the first and the second phases is 30 or 31.

40. A method for contraception of claim 33, wherein the first phase comprises 70 initial daily dosage units and the second phase comprises 14 daily dosage units.

41. A method for contraception of claim 33, wherein the first phase comprises 63 initial daily dosage units and the second phase comprises 7 daily dosage units.

42. A method for contraception of claim 33, wherein the competitive progesterone antagonist is selected from:
17α-ethinyl-17β-hydroxy-11β-(4-methoxyphenyl)estra-4,9-dien-3-one,
11β-(4-acetylphenyl)-17β-hydroxy-17α-(1-propinyl)estra-4,9-dien-3-one,
(Z)-11β-(4-acetylphenyl)-17β-hydroxy-17α-(3-hydroxy-1-propenyl)estra-4,9-dien-3-one,
11β-(4-dimethylaminophenyl)-17β-hydroxy-17α-(1-propinyl)estra-4,9-dien-3-one,
(Z)-9,11-dihydro-17β-hydroxy-17α-(3-hydroxy-1-propenyl)-6′-(3-pyridinyl)-4′H-naphth[3′,2′, 1′:10,9,11]estra-4,9(11)-dien-3-one,
(Z)-11β-[4-(dimethylamino)phenyl]-17β]-hydroxy-17α-(3-hydroxy-1-propenyl)-estr4-en-3-one,
4′,5′-dihydro-11′-[4-(dimethylamino)phenyl]-6β-methylspiro[estra-4,9-dien-17β,2′(3′H)-furan]-3-one,
4′,5′-dihydro-11β-[4-(dimethylamino)phenyl]-7β-methylspiro[estra-4,9-dien-17β,2′(3′H)-furan]-3-one, or
11β-(4-acetylphenyl)-19,24-dinor-17,23-epoxy-17α-chola-4,9,20-trien-3-one,
or a mixture thereof.

43. A method for contraception of claim 33, wherein the gestagen is selected from:
gestodene,
progesterone,
levonorgestrel,
cyproterone acetate,
chlormadinone acetate,
drospirenone (dihydrospirorenone),
norethisterone,
norethisterone acetate,
norgestimate,
desogestrel,
3-ketodesogestrel, or
dienogest,
or a mixture thereof.

44. A method for contraception of claim 33, wherein the daily dosage of gestagen is
0.02-0.6 mg of levonorgestrel,
0.02-2.0 mg of cyproterone acetate,
0.01-0.3 mg of gestodene,
0.02-0.3 mg of desogestrel
or a bioequivalent dosage of another gestagen.

45. A method for contraception of claim 33, wherein the gestagen gestodene is in a dosage of 0.02 to 0.075 mg.

46. A method for contraception of claim 33, wherein the competitive progesterone antagonist (Z)-11β-[4-(dimethylamino)phenyl]-17β-hydroxy-17α-(3-hydroxy-1-propenyl)estr-4-en-3-one is in a dosage of 0.01-5 mg.

47. A contraceptive kit of claim 29, wherein the separate subunits each contain 7 dosage units.

48. A contraceptive kit of claim 30, wherein the separate subunits of the first phase each contain 7 dosage units.

49. A contraceptive kit of claim 31, wherein the separate subunits of the first phase each contain 7 dosage units.
